(19) United States
(12) Patent Application Publication
(10) Pub. No.: US 2007/0141581 A1
(43) Pub. Date: Jun. 21, 2007

(54) Membrane androgen receptor as a therapeutic target for the prevention/promotion of cell death

(75) Inventors: Meharvan Singh, Benbrook, TX (US); Joshua W. Gatson, Fort Worth, TX (US)

Correspondence Address: CHALKER FLORES, LLP, 2711 LBJ FRWY, Suite 1036, DALLAS, TX, 75234, US

(73) Assignee: University of North Texas Health Science Center at Fort Worth, Fort Worth, TX (US)

(21) Appl. No.: 11/300,891

(22) Filed: Dec. 15, 2005

(51) Int. Cl. C12Q 1/68; G01N 33/567

(52) U.S. Cl. 435/6; 435/7.2

(57) ABSTRACT

The present invention includes compositions, kits and methods for specifically and differentially activating a membrane androgen receptor and their use for comparing the binding specificity of one or more drugs to a membrane androgen receptor and to an intracellular androgen receptor, wherein a difference in drug binding is indicative of differential receptor binding and may be used to diagnose and treat diseases and conditions associated with androgens.

### STATEMENT OF FEDERALLY FUNDED RESEARCH

This invention was made with U.S. Government support under Contract No. (AG22550 and AG23330), from the National Institutes on Aging, NARSAD-sponsored Young Investigator Award and the National Science Foundation SCORE (School's Cooperative Opportunities for Resources and Education) grant. The government has certain rights in this invention.

### TECHNICAL FIELD OF THE INVENTION

The present invention relates in general to the field of intracellular second messengers, and more particularly, to the use of a membrane androgen receptor as a therapeutic target for the modulation of cell function and viability.

### BACKGROUND OF THE INVENTION

Without limiting the scope of the invention, its background is described in connection with androgen therapy.

Androgens are classically associated with the regulation of muscle growth, spermatogenesis, growth of bone, and the development of secondary sexual characteristics in males and females (1, 2). Within the brain, testosterone can be aromatized to estradiol or alternatively, reduced at the 5α position to dihydrotestosterone (DHT) (1). DHT, however, is non-aromatizable and can elicit its effects via either genomic (classical) (1, 2) or non-genomic mechanisms (1, 3, 4). With regards to the latter, androgens have been shown to rapidly activate the phosphoinositide-3 (PI-3) kinase/Akt and/or mitogen-activated protein kinase (MAPK) pathways in a variety of peripheral tissues (3-5). However, whether these “non-genomic” effects are mediated by the classical androgen receptor or by some alternative mechanism remains controversial and unclear.

Two isoforms of the classical intracellular androgen receptor (AR) have been described, (AR-B and its N-terminally truncated form, AR-A), and are expressed in many different cell types (6-8). While the precise role of AR-A remains unclear, what has been described is that AR-A can antagonize the action of AR-B, a modulatory mechanism that may be relevant to the activation/inhibition of signaling pathways, regulation of gene transcription, as well as the regulation of cell survival (9). Alternatively, the existence of a plasma membrane receptor for androgens has also been proposed (18), like that described (or postulated) for estrogen and progesterone (11-17). The putative membrane AR has been described primarily in non-central nervous system tissue, including vascular tissue, macrophages, the ovary and in T cells (10, 19-24). Of interest is that this membrane-associated AR is linked to the activation of signaling pathways that may be important in regulating cell death, survival, or growth (18).

For years, androgen therapy has been provided to elderly men to help reduce bone loss, improve sexual function, and more recently, has been suggested to be of potential benefit in preventing brain dysfunction and neurodegenerative diseases, including Alzheimer's Disease. However, the literature is inundated with inconsistencies as to whether androgen treatment may be beneficial or detrimental. Further, androgen therapy, while providing potential benefit for the brain, may increase the risk for androgen-dependent neoplasms, including prostate cancer.

These inconsistencies prompted concern about the safety and efficacy of androgen therapy and led the NIH to ask the Institute of Medicine to review the literature on androgen replacement therapy and provide recommendations about the future prospects of androgen therapy. A major theme of the NIH analysis was that a more careful evaluation of the benefits and risks of androgen therapy is required.

Uses for steroids include those taught in U.S. patent application No. 20050153948, filed by Spilburg, C., for methods and formulations for the treatment of medical conditions related to elevated dihydrotestosterone. Briefly, the application describes a composition that contains a plant sterol or plant stanol or their fatty acid esters and an emulsifier for treating conditions that are related to elevated dihydrotestosterone. The compositions can be prepared in a dry form for use as a food ingredient, tablet or capsule. Alternatively, the compositions can be dissolved in oil.

Another example of the pursuit of steroid receptors is found in U.S. patent application No. 20050033018, filed by Lal, P.; et al., for receptors and membrane-associated proteins. The disclosure provides human receptors and membrane-associated proteins (REMAP) and polynucleotides that identify and encode REMAP. The invention also provides expression vectors, host cells, antibodies, agonists, and antagonists. The invention also provides methods for diagnosing, treating, or preventing disorders associated with aberrant expression of REMAP.

### SUMMARY OF THE INVENTION

The literature is replete with varying and contradictory reports of the effects of androgen therapy, with some reporting positive effects and many others reporting negative results under similar circumstances. Using the present invention, it is possible for the first time to sort out the different effects on specific cell-types, tissue and the like in each individual. It has been found that depending on whether the membrane androgen receptor or intracellular receptor is activated, the outcome will be different. As the skilled artisan will appreciate, under certain circumstances the membrane receptor may have a positive or negative outcome relative to intracellular receptor and vice versa. That is, activation or inactivation of one or the other receptor (membrane versus intracellular) may lead to a positive or negative outcome.

Furthermore, the skilled artisan will also recognize, in light of the current disclosure, that the mere presence or absence of the membrane, the intracellular or both receptors, are indicative of a change in the cellular status or homeostasis. For example, depending on the relative ratio of the membrane androgen receptor to the classical intracellular androgen receptor, the outcome consequent to androgen therapy may be different. Also, which of the receptors is activated, inactivated, blocked and the like will cause a variance in prognosis, diagnosis and/or therapy. Knowledge of androgen receptor status is a useful tool in predicting the therapeutic efficacy of androgens in a particular patient.

Using the compositions, methods, systems and kits disclosed herein, it is possible to rapidly and accurately detect, evaluate, quantitate, etc. the relative amounts of membrane and/or intracellular androgen receptor. One distinct advantage of the present invention is the ability to have an immediate, functional read-out for the relative ratio and/or activity of the two androgen receptors. Examples of the readouts are taught herein, including cell proliferation, apoptosis, phosphorylation and/or de-phosphorylation of certain proteins, activation or deactivation of certain second messengers (e.g., kinases and other intracellular signaling cascades), changes in the level of gene expression and the like.

In one embodiment, the present invention includes compositions and methods for selecting a drug candidate by comparing the binding specificity of one or more candidate drugs to a membrane androgen receptor and an intracellular androgen receptor, wherein a difference in drug candidate binding is indicative of differential receptor binding. The binding specificity may be measured by, e.g., flow cytometry, protein kinase activation, protein phosphorylation, gene expression, mRNA stability, protein expression, antibody binding, scintillation counting, cell count, cell division or cell apoptosis. The membrane androgen receptor may be measured using a non-internalized androgen bound to a non-internalizable agent, e.g., using a DHT-BSA, a DHT-bead, DHT-conjugated to a membrane-impermeable moiety, or a DHT-substrate. The intracellular androgen binding agent may be one or more androgen binding compound selected from testosterone, dihydrotestosterone, active metabolites of testosterone, and synthetic derivatives of testosterone such as testosterone propionate, testosterone cypionate, fluoxymesterone, flutamide and combinations thereof.

Using the present invention, the activation of the membrane androgen receptor can serve as a therapeutic target to modulate cell survival and/or cell death. In other embodiments, the activation of the membrane androgen receptor occurs independent of the intracellular androgen receptor. Using the compositions and methods taught herein, the inhibition of the membrane androgen receptor may be used to, e.g., promote the viability of brain cells. The method may also be used to determine, evaluate and use the relative ratio of the membrane androgen receptor to the classical intracellular androgen receptor, and the wherein the ratio of membrane to intracellular androgen receptor is used to predict and/or diagnose the outcome of androgen therapy. The method may also include the step of screening for the relative abundance of the membrane androgen receptor to identify patients that can receive androgen therapy safely versus those for which androgen therapy is contraindicated.

In another embodiment, the present invention include a kit with one or more vials that include: a non-internalizable androgen receptor binding agent, a labeled displaceable androgen and an intracellular androgen receptor binding agent, wherein the kit is used to measure the difference in the levels of a membrane androgen receptor and an intracellular binding receptor. Examples of non-internalizable androgen receptor binding agent includes: a C-19 steroid bound to a bead, an albumin-coated bead, an albumin protein, a charged chemical moiety, or a glass bead. The kit may also include a non-internalizable, labeled-displaceable androgen. Examples of labels for the displaceable androgen include, e.g., a radio-opaque material, a radioactive label, an enzymatic label, an epitope tag, a poly-His tag, a bead, a magnetic bead, a metal, or a fluoresent label such as 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, a Lanthanide Chelate, a Lanthanide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

In another embodiment, the present invention includes compositions and methods for diagnosing the relative health of a cell by comparing the ratio of membrane to intracellular androgen receptors, wherein a change in the ratio indicates a change in cellular homeostatis, e.g., metabolic status, cell-cycle status, functional status (e.g., production of signals, enzymes and the like) and the like. The method may be used for selecting patient for a clinical trial by determining a ratio of a membrane androgen receptor to an intracellular androgen receptor from a candidate patient sample; and optionally including or excluding the candidate patient from the trial if the ratio indicates a sensitivity to androgen therapy. The sensitivity of the patient may be due to the presence or absence (or relative ratio of) of intracellular androgen receptor activation, intracellular androgen receptor inactivation, membrane androgen receptor activation, membrane androgen receptor inactivation and combinations thereof. In some instances, the patient may be suspected of having a disease associated with or related pathogenetically to androgen receptor activation and wherein the patient is tested for the ratio of preferential binding of a candidate agent to a membrane androgen receptor or an intracellular androgen receptor.

Yet another embodiment of the present invention includes a method for identifying one or more candidate agents from a pool of agents by detecting the effect of one or more agents from a pool of agents on membrane androgen receptor binding, wherein a change in the level of binding is indicative of competition of the membrane androgen receptor and is used to further identify the candidate agents. The step of detecting the binding of the one or more candidate agents may be binding to one or more intracellular androgen receptors and/or detecting the binding of the one or more candidate agents to the intracellular androgen receptor binding and selecting further those candidate agents that have higher bind to the membrane androgen receptor than to the intracellular androgen receptor.

Another embodiment of the present invention is a method for treating a disease associated with or related pathogenetically to androgen receptor activation by preferentially binding a membrane androgen receptor or an intracellular androgen receptor. The membrane androgen receptor may be preferentially activated over the intracellular androgen receptor or the intracellular androgen receptor may be preferentially activated over the membrane androgen receptor. In some embodiments, the disease is an age-related disease that causes brain cell apoptosis or inactivation, e.g., Parkinson's disease, Alzheimer's disease, a brain cancer, affects glial cells, astrocytes, cortical cells, hippocampal neurons and the like.

The present invention also includes a composition for treating a disease associated with or related pathogenetically to androgen receptor activation in which the composition binds preferentially to a membrane androgen receptor or an intracellular androgen receptor. Examples of composition that bind preferentially to a membrane androgen receptor include, e.g., DHT-BSA, a DHT-bead, DHT-conjugated to a membrane-impermeable moiety, a DHT-substrate and combinations thereof. While DHT is specifically set forth in this example, the skilled artisan will readily recognize that the androgen receptor binding portion (DHT) may be substituted by any of a number of other binding agonists or antagonists, e.g., testosterone, dihydrotestosterone, active metabolites of testosterone, and synthetic derivatives of testosterone such as testosterone propionate, testosterone cypionate, fluoxymesterone, flutamide and combinations thereof.

Another embodiment of the present invention is a method for treating brain cells associated with or related pathogenetically to androgen receptor activation by preferentially binding an active agent to a membrane androgen receptor. For example, the active agent may be a protein, nucleic acid, carbohydrate, lipid, fatty acid, bead, a membrane-impermeable moiety, a substrate and combinations thereof conjugated to a testosterone, dihydrotestosterone, active metabolites of testosterone, and synthetic derivatives of testosterone such as testosterone propionate, testosterone cypionate, fluoxymesterone, flutamide. The active agent may include both an intracellular androgen receptor binding agent selected from testosterone, dihydrotestosterone, active metabolites of testosterone, and synthetic derivatives of testosterone such as testosterone propionate, testosterone cypionate, fluoxymesterone, flutamide and combinations thereof; and a membrane androgen receptor binding agent selected from a protein, nucleic acid, carbohydrate, lipid, fatty acid, bead, a membrane-impermeable moiety, a substrate and combinations thereof conjugated to a testosterone, dihydrotestosterone, active metabolites of testosterone, and synthetic derivatives of testosterone such as testosterone propionate, testosterone cypionate, fluoxymesterone, flutamide, wherein the ratio of the membrane versus intracellular androgen receptor binding agent is modulated to treat the disease.

Yet another embodiment of the present invention includes a composition for treating a disease associated with or related pathogenetically to a membrane-associated androgen receptor by providing a pharmaceutically effective amount of an androgen receptor binding agent conjugated to a moiety that prevents or reduces the entry of the composition into a cell. Examples of moieties that prevent or reduce the entry of the composition into the cell may be selected from, e.g., a protein, lipid, fatty acid, carbohydrate, a nucleic acid, a charged alkyl group, and combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 is a Western Blot which shows that the classical androgen receptor is expressed in C6 glial cells;

FIG. 2 is a graph and Western Blot which shows that the DHT-induced ERK phosphorylation is mediated by the classical AR;

FIG. 3 is a graph and Western Blot which shows that DHT elicits an increase in phospho-Akt levels in C6 glial cells;

FIG. 4 is a graph and Western Blot which shows that DHT/BSA treatment suppresses phospho-ERK levels in C6 glial cells;

FIG. 5 is a graph and Western Blot which shows that DHT/BSA suppression of phospho-ERK levels in C6 glial cells is Flutamide insensitive;

FIG. 6 is a graph and Western Blot that shows that shows that carboxymethyloxime (CMO) conjugation to DHT does not alter DHT's ability to elicit ERK phosphorylation;

FIG. 7 is a graph and Western Blot which shows that BSA does not elicit ERK phosphorylation;

FIG. 8 a graph and Western Blot which shows that DHT-BSA treatment suppresses phospho-Akt levels in C6 glial cells;

FIG. 9 a graph and Western Blot which shows that DHT-BSA blocks DHT-induced ERK phosphorylation;

FIG. 10 is a bar graph that shows that DHT-BSA exacerbates iodoacetic acid (IAA) induced cell death of C6 glial cells; and

FIG. 11 is flow-cytometry data which shows that DHT-BSA binds to specific sites on the cell surface of C6 glial cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The androgens are steroids that develop and maintain primary and secondary male sex characteristics. Androgens are derivatives of cyclopentanoperhydrophenanthrene. Endogenous androgens are C-19 steroids with a side chain at C-17, and with two angular methyl groups. Testosterone is the primary endogenous androgen. Methyltestosterone is a synthetic derivative of testosterone suitable for oral administration. Androgens suitable for use in methods of the present invention include, e.g., testosterone, dihydrotestosterone, active metabolites of testosterone, and synthetic derivatives of testosterone such as testosterone propionate, testosterone cypionate, and fluoxymesterone.

Androgen antagonists block the synthesis or action of androgens. Compounds that inhibit testosterone synthesis include GnRH agonists such as leuprolide or gonadorelin (Goodman and Gilman, The Pharmacological Basis of Therapeutics, Ninth Edition, p. 1453 (1996)). Androgen-receptor antagonists inhibit the binding of androgen to its receptor. Various androgen antagonists are known in the art, including cyproterone acetate, flutamide, nilutamide, and bicalutamide. See also, U.S. Pat. No. 5,593,981; U.S. Pat. No. 5,610,150; U.S. Pat. No. 4,161,540; U.S. Pat. No. 3,995,060.

Androgens for use with the methods of the present invention may include natural and artificial androgens, e.g., endogenous androgens, testosterone and 5α-dihydrotestosterone and modifications thereof. If desired, it is possible to use any of the wide variety of synthetically made androgens to determine receptor specificity. Any of the wide range of steroidal and non-steroidal compositions that mimic the effect of endogenous androgens may be used, including those that are antagonists. The androgen of choice to be used within the in-vitro methods should be highly potent and be without antagonistic properties. One example is 5α-dihydrotestosterone (hereinafter “5α-DHT”), however, testosterone itself may be employed. Both are recognized as potent endogenous androgens.

As used herein, the terms “membrane androgen receptor binding” or “membrane androgen receptor impermeable moiety” refer to those agents or compounds that include all those androgens and derivatives that are excluded, in whole or in part, from entry into the cell but that would otherwise bind to either the intracellular androgen receptor or the putative membrane androgen receptor. Examples of those androgens that selectively bind the membrane androgen receptor are all the known androgens that are derivatives of cyclopentanoperhydrophenanthrene, e.g., endogenous androgens are C-19 steroids with a side chain at C-17, and with two angular methyl groups, testosterone, methyltestosterone, dihydrotestosterone, active metabolites of testosterone, and synthetic derivatives of testosterone such as testosterone propionate, testosterone cypionate and fluoxymesterone, to name a few, that have been modified to prevent or reduce their entry into the cell by, e.g., conjugating them to a protein or other moiety, e.g., a bead, a large protein (e.g., albumin), nucleic acid, lipid, fatty acid, carbohydrate, charged molecule (from atoms to particles), beds, glass, quartz, silicon, polymers, multimers, oligomers, metals, nanoparticles, microparticles, etc.

The moieties that prevent or reduce intracellular transportation will generally be biocompatible and may even be biodegradable, conductive, magnetic or ferrous. Examples of beads that may be used that are biocompatible and acceptable for human use include those made from carbohydrates, such as natural sources. Examples of proteins that are biocompatible and that generally do not cross cell membranes include lymphokines, cytokines, structural and scaffold proteins (e.g., collagen, cartilage, fibronectin), combinations of glycol and lipid containing proteins (e.g., LDL, VLDL, HDL, and subcomponents thereof, etc.), enzymes (e.g., alkaline phosphatase, metabolic enzymes, complement cascade proteins, clotting cascade proteins), transport proteins (e.g., hemoglobin, albumin), receptors (e.g., lectins, lympo- and cytokine receptors), transport proteins (e.g., glucose transporter), and the like.

As used herein, the term “brain cell” refers to those cells that are found in, about or associated with cells of the central nervous system and the brain, including, the lower, mid and upper cortex, immune cells and support cells associated therewith. Brain cells include all types of neurons, e.g., afferent neurons, efferent neurons, and interneurons, whether pseudounipolar, bipolar, multipolar and the like. Cells in the brain include glial cells, astrocytes, Schwann cells, Purkinje cells, and the like as will be known to the skilled artisan.

For example, the conjugated androgen may be conjugated to an agent or moiety that targets the overall molecule to the “lipid compartment” of a cell. As used herein, the phrase “lipid compartment” refers to compounds that have cyclic or acyclic long-chain aliphatic hydrocarbons and their derivatives, such as fatty acids, alcohols, amines, amino alcohols and aldehydes. For example, common lipids include fatty acids, fats, phospholipids, steroids, eicosanoids, waxes and fat-soluble vitamins. Some lipids may be generally classified into two groups, the simple lipids and the complex lipids, e.g., triglycerides or fats and oils, fatty acid esters of glycerol, waxes, fatty acid esters of long-chain alcohols and steroids such as cholesterol and ergosterol. Complex lipids include, e.g., phosphatides or phospholipids (phosphorous containing lipids), glycolipids (carbohydrate containing lipids), and sphingolipids (sphingosine containing lipids). The portion of the molecule that includes the delivery agent may even make the overall molecule compatible with oral administration.

As used herein, the term “lipid” includes fats or fat-like substances. The term is descriptive rather than a chemical name such as protein or carbohydrate. Lipids include true fats (i.e., esters of fatty acids and glycerol), lipoids (i.e., phospholipids, cerebrosides, waxes) and sterols (i.e., cholesterol, ergostrol) that may be conjugated, covalently or non-covalently to the androgen to prevent or reduce its rate of entry into a cell and/or increase its half-life in association with the membrane androgen receptor. Lipids can be a target of oxidation through mechanisms, such as autoxidation. As used herein, the term “fatty acid” refers to a group of, e.g., negatively charged, generally linear hydrocarbon chains. The hydrocarbon chains of fatty acids vary in length and oxidation states. Generally, fatty acids have a negatively charged portion (e.g., at the carboxyl end), and a “tail” portion, which determines the water solubility and amphipathic characteristics of the fatty acid. For example, fatty acids are components of the phospholipids that include biological membranes, as fats, which are used to store energy inside cells, or for transporting fat in the bloodstream. As used herein, the term “phospholipid” refers to any of the class of esters of phosphoric acid that include at least one of the following side-groups: a fatty acid, an alcohol and a nitrogenous base.

As used herein, the term “fat” or “fats” refers to any of the glyceryl esters of fatty acids, e.g., the monoacylglycerol, diacylglycerol and triacylglycerol forms of fatty acids that may be conjugated, covalently or non-covalently to the androgen to prevent or reduce its rate of entry into a cell. Triglycerides refer to those molecules that are neutrally charged and entirely hydrophobic, i.e., reduced molecules. Monoacylglycerides and diacylglycerides are metabolic intermediates in phospholipid synthesis, while triglycerides form the fat molecules that are used to store chemical energy in a water free, compact state. As used herein, the term “fat-soluble vitamins” refers to, e.g., common fat-soluble vitamins include vitamin (A) (retinol), Vitamin D (e.g., vitamin D3 (cholecalciferol)), Vitamin E, Vitamin K and the like.

A large variety of carbohydrates may be used to prevent entry of an androgen into the cell, e.g., those if natural and synthetic sources such as shrubs, trees, plants, yeasts, fingi, molds, gums, resins, starch and cellulose derivatives and natural mucin sources. Specifically, some of the natural sources include: (a) shrub or tree exudates which contain acacia, karaya, tragacanth, or ghatti; (b) marine gums which include agar, algin, or carrageenan; (c) seed gums which include guar, locust bean, or psyllium; (d) plant extracts which contain pectins or acetylated polymannose; (e) starch and cellulose derivatives such as hetastarch, carboxymethylcellulose, ethylcellulose, hydroxypropyl methylcellulose, methylcellulose, oxidized cellulose; and microbial gums which contain dextrans, xanthan. However, it should be recognized that the composition of the invention is not intended to be limited by the source from which the respective carbohydrates are obtained.

As used herein, the term “carbohydrate” is used interchangeably with the terms “saccharide,” “polysaccharide,” “oligosaccharide” and “sugar” the definitions of which are well known to those skilled in the art of carbohydrate chemistry. Carbohydrate compositions for use with the present invention may be selected and customized to provide controlled delivery of the androgen to the membrane depending on the nature of the bonds between the individual monomers, the types of monomer and any other modification (e.g., myristilation, conjugation to a protein or other non-degradable agent).

The membrane androgen receptor-specific binding agents may be contacted to cells, in vitro or in vivo, in a variety of dosage forms. For example, the membrane androgen receptor-specific binding agents may be provided to a patient through a variety of locations, e.g., oral, intravenous (bolus or infusion), intraperitoneal, subcutaneous, intramuscular, pulmonary, intradural, intrarenal, percutaneous, and the like in a form adapted for such delivery as is well known to those of ordinary skill in the pharmaceutical arts.

Dosage forms. A dosage unit for use of the membrane androgen receptor-specific binding agents of the present invention may be a single compound or mixtures thereof. For example, the agent may be included with other compounds such as a potentiator or counter-activator (e.g., an antagonist of the intracellular androgen receptor). The compounds may be mixed together, form ionic or even covalent bonds. The membrane androgen receptor-specific binding agents of the present invention may be administered in oral, intravenous (bolus or infusion), intraperitoneal, subcutaneous, intrapulmonary, intramuscular form, and the like, using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the membrane androgen receptor-specific binding agents of the present invention to a patient in need of therapy that includes, alone in combination, an agent that causes: intracellular androgen receptor activation, intracellular androgen receptor inactivation, membrane androgen receptor activation, membrane androgen receptor inactivation and combinations thereof.

The membrane androgen receptor-specific binding agents may also be administered as any one of known salt forms. Membrane androgen receptor-specific binding agents are typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the membrane androgen receptor-specific binding agents may be formulated to provide, e.g., maximum and/or consistent dosing for the particular form for oral, rectal, topical, intravenous injection or parenteral administration. While the membrane androgen receptor-specific binding agents may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.), and the like, relevant portions incorporated herein by reference.

For example, the membrane androgen receptor-specific binding agents may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

Membrane androgen receptor-specific binding agents may also be administered in the form of liposome delivery systems, e.g., small unilamellar vesicles, large unilamallar vesicles, and multilamellar vesicles, whether charged or uncharged. Liposomes may include one or more: phospholipids (e.g., cholesterol), stearylamine and/or phosphatidylcholines, mixtures thereof, and the like. Membrane androgen receptor-specific binding agents may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the membrane androgen receptor-specific binding agents may be coupled one or more biodegradable polymers to achieve controlled release of the membrane androgen receptor-specific binding agents, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, capsule or gelatin capsules (gelcaps) may be loaded with the membrane androgen receptor-specific binding agents and one or more powdered carriers or fillers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagous, tachea, lungs and alveoli, the membrane androgen receptor-specific binding agents may also be delivered as an intranasal form via use of a suitable intranasal vehicle. Generally, the smaller the particle the deeper the delivery, as such, the membrane androgen receptor-specific binding agents may be prepared into nanoparticles by, e.g., freeze-spraying, to form individual nanoparticles. For dermal and transdermal delivery, the membrane androgen receptor-specific binding agents may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of membrane androgen receptor-specific binding agents may include the following forms.

Capsules. Capsules may be prepared by filling standard two-piece hard gelatin capsules each with, e.g., 10 to 500 milligrams of powdered active ingredient (e.g., membrane androgen receptor-specific binding agent(s)), 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. For example, the active ingredient is prepared and injected by using a positive displacement pump into gelatin to form soft gelatin capsules containing, e.g., 1-500 milligrams of the membrane androgen receptor-specific binding agents. The capsules are washed and dried.

Tablets. A large number of tablets are prepared by conventional procedures so that the dosage unit was, e.g., 10-500 milligrams of membrane androgen receptor-specific binding agent(s), 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by, e.g., stirring 0.1 to 1.5% by weight of membrane androgen receptor-specific binding agent(s) in deionized water (or other solvent) and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 include, e.g., 1-500 mg of the membrane androgen receptor-specific binding agent(s), 200 mg of sodium carboxymethyl cellulose, 5 mg of sodium benzoate, 1.0 g of sorbitol solution, U.S.P., and 0.025 ml of vanillin. For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of brain cells, which include one or more containers containing a pharmaceutical composition with a therapeutically effective amount of membrane androgen receptor-specific binding agents. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

The present inventors have determined that a lack in the complete understanding of the biology of androgens is a key roadblock in the ability to safely and efficiently identify, develop and modify androgen agonists, antagonists and derivatives thereof, for use in safer and more efficacious therapies. The existence of androgen binding sites on the plasma membrane of non-neural (or non-brain) cells has been shown by others and the effect of activating this putative membrane androgen receptor is to promote apoptosis. However, those studies were not able to compare the consequences of activating the membrane androgen receptor with that resulting from activating the intracellular receptor.

While the existence of testosterone binding sites on the plasma membrane has been shown by binding this putative entity remains as yet uncloned. The present invention is the first to demonstrate the important and clinical relevance of activating the membrane androgen receptor, which results in an effect that is functionally distinct from activating the intracellular androgen receptor. The present inventors have recognized and demonstrate herein that the membrane androgen receptor is an important biological substrate. The model and observations made herein provide a method and compositions for the rapid, functional evaluation, characterization and modification of drugs that target the membrane androgen receptor.

It is shown herein that the consequences of activating the membrane androgen receptor versus the classical intracellular receptor can be exploited for the purposes of screening those individuals who are at risk when undergoing androgen therapy versus those individuals who would be appropriate candidates for androgen therapy.

The present invention was used to identify the source of the discrepancy in the literature and provides compositions, methods and kits for rapidly detecting and determining whether an androgen-based therapy will be safe and effective or deleterious. The present invention may be used to detect, evaluate, compare and contrast the presence or absence of two competing pathways through which androgens, including testosterone, dihydrotestosterone and its analogues, elicit their effects. One of these mechanisms is elicited through a “membrane” androgen receptor. In one model system, the present invention may be used to determine functionally the presence, absence and/or competing effect of the non-internalized androgen receptor to promote cell death, whereas activation of the other receptor mechanism (the “classical intracellular androgen receptor”) did not promote cell death.

Thus, the relative abundance of one receptor type over another may shift the balance of how androgens influence cell viability. Specifically, if a particular disease or insult leads to an increased expression of the “membrane androgen receptor”, this would promote androgen-induced cell death, even if the level of circulating (blood) androgen itself were not modified. Thus, the membrane androgen receptor can serve as a therapeutic target to modulate cell survival and/or cell death. In the brain, inhibition of the membrane androgen receptor would promote the viability of brain cells. Importantly, the ability to screen for the relative abundance of the membrane androgen receptor (using the method described herein) would enable the identification of the appropriate population of men that can receive androgen therapy safely versus those for which androgen therapy is contraindicated.

The present invention includes a new cellular target, compositions, methods, kits and systems for the determination, isolation, characterization, evaluation, improvement of drugs that may have profound consequences for protecting, e.g., brain cells. The present invention includes compositions, kits and methods for screening for those individuals that androgen therapy is appropriate and those individuals for whom androgen therapy is contraindicated in that androgens may exacerbate or accelerate the progression of brain disorders. One particular use for the present invention is the prognosis and evaluation of patients before entering them in a clinical trial, that is, the present invention may be used to select pre-select and/or protect patients from entering a clinical trial. Furthermore, the present invention may be used to evaluate patients that have caused inconsistencies in the literature and is useful for the safer use and more effective treatments that involve androgens.

The present inventors have recognized that one of these mechanisms is elicited through a putative membrane androgen receptor, and promotes cell death, whereas activation of the other receptor mechanism (the “classical intracellular androgen receptor”) does not. Thus, the relative abundance of one receptor type over another may shift the balance of how androgens influence cell viability. Specifically, if a particular disease or insult leads to an increased expression of the “membrane androgen receptor”, this would promote androgen-induced cell death, even if the level of circulating (blood) androgen itself were not modified.

It was further discovered a new putative cellular target of future drug development that may have profound consequences for protecting brain cells, or alternatively, serving as the basis for screening for those individuals that androgen therapy is appropriate and those individuals for whom androgen therapy is contraindicated in that androgens may exacerbate or accelerate the progression of brain disorders. This discovery also resolves the inconsistencies in the literature and will lead to safer and more effective treatments that involve androgens.

The role of the classical AR in the “non-genomic” effects of androgens on glia, was evaluated using DHT and a membrane impermeable, BSA-conjugated androgen (DHT-BSA), on the phosphorylation of ERK and Akt, two key effectors within the MAPK and PI-3K signaling pathways, respectively. It was found that DHT induced the phosphorylation of ERK and DHT-BSA resulted in a dose-dependent suppression of both ERK and Akt phosphorylation. DHT-BSA also blocked the effects of DHT. As the DHT-BSA was not able to cross the membrane, the DHT-BSA must act upon a novel membrane-associated AR (or like receptor), which was further supported by the identification of DHT-displaceable binding sites on the cell surface of live C6 glial cells. Therefore, the present invention allows for the further identification and characterization of a novel plasma membrane-associated androgen receptor and permits, for the first time, to allow for the evaluation and detection of agents that are able to affect one or both competing pathways, within a specific cell or tissue type.

Materials and Methods. Cell culture. Rat glioma cells (C6; American Type Culture Collection, Manassas, Va.) were propagated in Dulbecco's Modified Eagle Medium (DMEM; Invitrogen, Carlsbad, Calif.) supplemented with 10% charcoal-stripped fetal bovine serum (FBS; Hyclone, Logan, Utah), and maintained at 37° C. in a humidified environment containing 5% CO₂.

Following treatment of the cells with the appropriate duration and dose of hormone, the cells were harvested, homogenized, and centrifuged and the supernatant was subsequently collected and analyzed for total protein concentration using the Biorad DC protein assay kit. Pre-prepared cell lysates from the prostate cancer cell line, LNCaP, (Santa Cruz Biotechnology, Santa Cruz, Calif.) were obtained and used as positive controls for the detection of the androgen receptor.

Treatment of cultures. C6 glial cells were treated with either vehicle control (DMSO, 0.1%), dihydrotestosterone (DHT) (Steraloids Inc., Newport, R.I.), or 5α-androstan-17β-ol-3-one-3-o-carboxymethyloxime:BSA (DHT-BSA) (Steraloids Inc.) at the concentrations indicated for 30 minutes, to assess the effects of these hormones on ERK and Akt phosphorylation. Controls for the BSA-conjugated DHT (DHT-BSA) treatment included treatment with equimolar concentrations of 5α-androstan-17β-ol-3-one-3-o-carboxymethyloxime (DHT:CMO) (Steraloids Inc.) or bovine serum albumin (BSA) alone (Fisher Scientific, Fair Lawn, N.J.) for a similar duration (30 min). Inhibition of the classical androgen receptor was achieved using the androgen receptor antagonist, Flutamide (3 μM; Sigma-Aldrich, St. Louis, Mo.), which was applied 30 minutes prior to treatment with the hormone. Inhibition of MEK, the signaling protein upstream of ERK was achieved using U0126 (10 μM; Cell Signaling, Beverly, Mass.), also pre-incubated for a period of 30 minutes prior to hormone administration.

Western blot analysis. Following treatment with hormone and/or inhibitor, C6 glial cells were harvested into lysis buffer containing protease and phosphatase inhibitors as described previously (25). Following homogenization, samples were centrifuged at 99,000×g for 15 min at 4° C., and the resulting supernatants were evaluated for total protein concentrations using the BioRad DC (Biorad Labs, Hercules, Calif.) protein assay kit [based on the method of Lowry; (26)]. Sample lysates were loaded onto a sodium dodecyl sulfate (SDS), 10% polyacrylamide gel (PAGE), subjected to electrophoresis, and subsequently transferred onto a polyvinylidene difluoride membrane (PVDF; 0.22 μm pore size, BioRad). The membrane was blocked for 6 hrs with a 3% BSA in 0.2% Tween-containing TBS (TBS-T) solution prior to application of the primary antibody. The following primary antibodies were used: for the detection of the androgen receptor, anti-AR (C-19) (1:200; Santa Cruz Biotechnology); for the detection of the phosphorylated form of Akt: rabbit anti-phospho-Akt (Ser473; 1:1000; Cell Signaling, Beverly, Mass.), for the detection of total Akt, anti Akt (1:1000; Cell Signaling); for the detection of the phosphorylated form of ERK1/2, rabbit anti phospho-p44/42 Map Kinase (Thr202/Tyr204, 1:1000; Cell Signaling); for the detection of total ERK1 and ERK2, goat anti-ERK1 (C-16, 1:500)/goat anti-ERK2 (C-14, 1:500; Santa Cruz Biotechnology). Antibody binding to the membrane was detected using a secondary antibody (either goat anti-rabbit or rabbit anti-goat) conjugated to horseradish peroxidase (1:20,000; Pierce, Rockford, Ill.) and visualized using enzyme-linked chemiluminescence (ECL, Amersham, Arlington Heights, Ill.) with the aid of the UVP imaging system. Phospho-Akt and phosphoERK blots were re-probed with anti-Akt or anti ERK1/2 antibodies to ensure equal loading across lanes.

Flow cytometry. Briefly, C6 glial cells (10⁶ cells) were pipetted into a 1.5 ml micro-centrifuge tube, centrifuged at 250×g for 5 minutes and washed twice with 1 ml of PBS. After the last wash, the cells were re-pelleted, and suspended in 100 μl of PBS and treated with DHT-BSA-FITC (50 μM; Sigma Aldrich) in the presence or absence of dihydrotestosterone (DHT; 1 mM), for 30 minutes at 4° C. In parallel, cells were re-suspended in PBS treated with BSA-FITC (50 μM; Sigma Aldrich), serving as the control for the detection of “non-specific binding”. Following this incubation period, the cells were washed twice in PBS and resuspended in 500 μl of PBS. The labeled C6 glial cells were injected into an EPICS XL-MCL flow cytometer (Beckman Coulter) and analyzed with System 2 software (Beckman Coulter). Graphical representation of the data was generated using the FloJo software (Tree Star, Inc., San Carlos, Calif.).

Statistical Analysis. Densitometric analysis of the Western blots was conducted using the LabWorks Image Acquisition and Analysis Software (UVP Inc., Upland, Calif.). Densitometric data from at least three independent studies was subjected to an analysis of variance (ANOVA), followed by a Tukey's post hoc analysis for the assessment of group differences, and presented as a bar graph depicting the average±S.E.M., using the GraphPad Software (San Diego, Calif.).

C6 glial cells express the androgen receptor (AR). Genomic and non-genomic pathways activated by androgens may involve one or both receptor isoforms of the androgen receptor (AR-B or AR-A). Using Western blot analysis, the inventors evaluated whether the C6 glial cells express either of these receptor isoforms.

FIG. 1 shows that the “classical” and/or “intracellular” androgen receptor is expressed in C6 glial cells. Total protein from C6 glial cells was isolated and evaluated for the expression of the AR using Western blot analysis. This analysis revealed two bands corresponding to the predicted molecular weights of AR-B (110 kDa) and AR-A (87 kDa). These bands co-migrated with the AR bands observed in the prostate cancer cell line, LNCAP, serving as the positive control. FIG. 1 confirms the presence of immunoreactive AR, and identifies two distinct bands corresponding to molecular weights of 110 and 87 kDa, respectively. These two bands are of the same size described in the literature for the full length, AR-B and the truncated, AR-A. Moreover, these bands co-migrated with the AR-B and AR-A bands seen in the positive control, a cell lysate derived from the prostate cancer cell line, LNCaP cells.

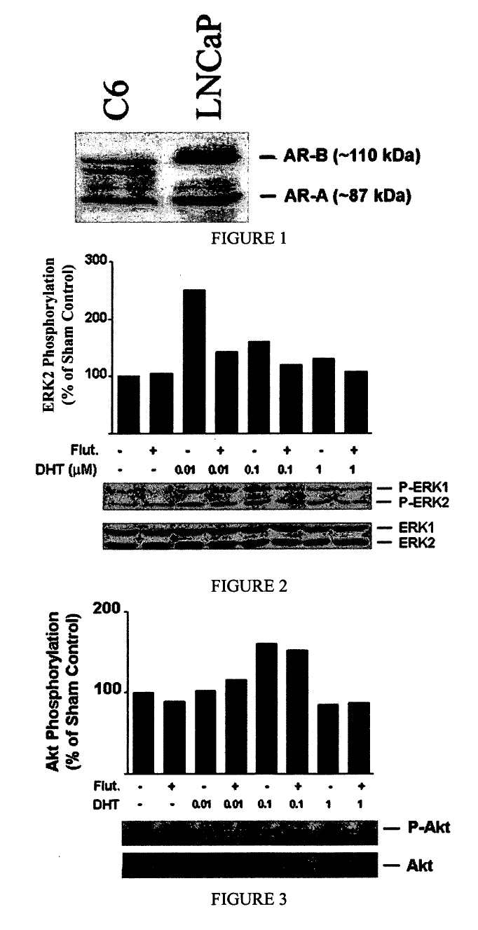

The nuclear AR mediates DHT-induced phosphorylation of ERK. Given that androgens have been shown to elicit activation of cell signaling pathways in a variety of tissues, it was determined whether DHT elicits the phosphorylation of ERK, a key effector of the MAPK pathway, in glia. Treatment of C6 glial cells with 10 nM DHT resulted in a robust (2.5-fold) increase in the phosphorylation of ERK. This effect was inhibited by the classical androgen receptor antagonist, Flutamide (FIG. 2).

FIG. 2 shows that the effect of DHT-induced ERK phosphorylation is mediated by the classical AR. C6 glial cells were treated with DHT (0.01, 0.1, 1 μM) for 30 min in the presence/absence of the AR antagonist Flutamide (Flut., 3 μM). Resulting lysates (containing 60 μg of total protein) were then subjected to SDS-PAGE and Western blot analysis for the evaluation of ERK phosphorylation. The results demonstrate that DHT-elicits ERK phosphorylation, an effect that was blocked by the classical AR antagonist, flutamide. The upper blot and lower blot depicts ERK phosphorylation and total ERK protein, respectively, from a single independent experiment. The bar graph (upper panel), however, represents the densitometric analysis from two independent studies.

The effect of DHT on ERK phosphorylation was also blocked by the MEK1/2 inhibitor, U0126 (data not shown), suggesting that the effect of DHT on ERK required the activation of the upstream signaling kinase, MEK. Interestingly, higher concentrations of DHT (0.1 and 1 μM) did not result in an increase in ERK phosphorylation (FIG. 2). DHT also induced an increase in Akt phosphorylation, but required a slightly higher concentration than that which was required to elicit ERK phosphorylation. Another distinction was that the antagonist to the classical androgen receptor, flutamide, failed to inhibit the effect of DHT on Akt phosphorylation (FIG. 3).

FIG. 3 shows that DHT elicits an increase in phospho-Akt levels in C6 glial cells. Rat glioma cells were treated with DHT (0.01, 0.1, 1 μM) for 30 min in the presence/absence of the AR antagonist Flutamide (Flut., 3 μM). Resulting lysates (containing 60 μg of total protein) were then subjected to SDS-PAGE and Western blot analysis for the evaluation of Akt phosphorylation. The results show that DHT elicited an increase in Akt phosphorylation, but was not inhibited by the AR antagonist, flutamide. The upper blot and lower blot depicts Akt phosphorylation and total Akt protein, respectively, from a single independent experiment. The bar graph (upper panel), however, represents the densitometric analysis from two independent studies and is presented as signal intensity relative to that seen in the sham (vehicle-treated) control.

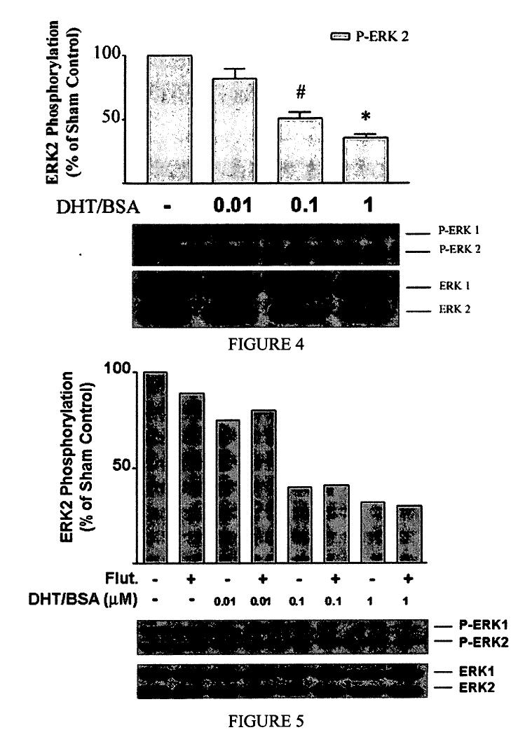

Activation of a membrane AR decreased the phosphorylation of ERK. In an attempt to assess if the effect of DHT was mediated by the classical intracellular/intranuclear androgen receptor or alternatively, if a membrane androgen receptor may be involved, the inventors determined whether the membrane-impermeable androgen, DHT-BSA, would also elicit ERK phosphorylation. In contrast to the effect of DHT, the membrane-impermeable androgen not only failed to elicit an increase in the phosphorylation of ERK, but resulted in a substantial suppression of ERK phosphorylation, particularly at the higher concentrations of 100 nM and 1 μM (FIG. 4).

FIG. 4 shows that the effect of DHT/BSA treatment is to suppress phospho-ERK levels in C6 glial cells. C6 glial cells were treated with increasing concentrations of DHT-BSA (0.01, 0.1, and 1.0 μM) for 30 min. Resulting lysates (containing 60 μg of total protein) were then subjected to SDS-PAGE and Western blot analysis for the evaluation of ERK phosphorylation. DHT-BSA resulted in a dose-dependent decrease in phospho-ERK levels. The data revealed that DHT-BSA inhibited basal ERK phosphorylation levels in a dose-dependent manner. The upper blot and lower blot depict ERK phosphorylation and total ERK protein, respectively, from a single independent experiment. The bar graph (upper panel), however, represents the densitometric analysis and statistical evaluation of data from three independent studies, and is presented as signal intensity relative to that seen in the sham (vehicle-treated) control. Statistical significance was determined using a one-way ANOVA, followed by Tukey's post-hoc analysis for group differences (*=p<0.001; #=p<0.01). This suppression of ERK phosphorylation by DHT-BSA was also insensitive to Flutamide (FIG. 5).

FIG. 5 shows that DHT/BSA suppressed phospho-ERK levels in C6 glial cells and that the effect is not sensitive to Flutamide. C6 glial cells were treated with increasing concentrations of DHT-BSA (0.01, 0.1, and 1.0 μM) for 30 min. in the presence/absence of the AR antagonist flutamide (Flut., 3 μM). Resulting lysates (containing 60 μg of total protein) were then subjected to SDS-PAGE and Western blot analysis for the evaluation of ERK phosphorylation. The results demonstrated that the dose-dependent inhibition of ERK phosphorylation by DHT-BSA was not prevented by the AR antagonist, flutamide. The upper blot and lower blot depict ERK phosphorylation and total ERK protein, respectively, from a single independent experiment. The bar graph (upper panel), however, represents the densitometric analysis from two independent studies, and is presented as signal intensity relative to that seen in the sham (vehicle-treated) control.

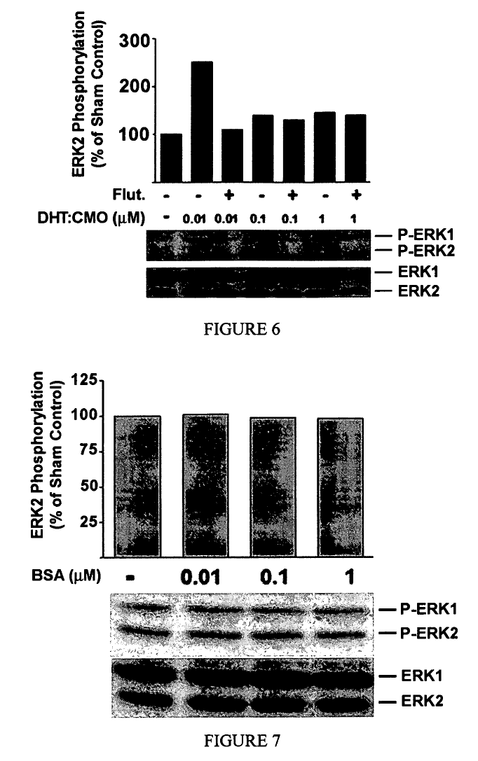

To ensure that the inhibition of ERK was not due to the chemical modification of the parent DHT molecule, the effect of DHT:CMO (5α-androstan-17β-ol-3-one-3-o-carboxymethyloxime) was evaluated. As expected, the carboxymethyloxime (CMO) group alone did not alter the ability of the androgen to elicit ERK phosphorylation (FIG. 6), and recapitulated the data seen with DHT alone (FIG. 2). Further, the effect of DHT:CMO on ERK phosphorylation was also blocked by the androgen receptor antagonist, flutamide (FIG. 6).

FIG. 6 shows that Carboxymethyloxime (CMO) conjugation to DHT does not alter DHT's ability to elicit ERK phosphorylation. C6 glial cells were treated with DHT:CMO (0.01, 0.1, 1 μM) for 30 min in the presence/absence of the AR antagonist Flutamide (3 μM). DHT:CMO was included in these studies as a control group for the BSA-conjugated DHT (DHT-BSA). Resulting lysates (containing 60 μg of total protein) were then subjected to SDS-PAGE and Western blot analysis for the evaluation of ERK phosphorylation. The results show that the CMO moiety does not alter the ability of DHT to elicit ERK phosphorylation. The upper blot and lower blot depicts ERK phosphorylation and total ERK protein, respectively, from a single independent experiment. The bar graph (upper panel), however, represents the densitometric analysis from two independent studies, and is presented as signal intensity relative to that seen in the sham (vehicle-treated) control.

As an added control, the inventors determined whether the inhibitory effect of DHT-BSA could have been attributed to the bulky globulin (BSA) that was attached. Administration of BSA by itself failed to alter the basal phosphorylation state of ERK, suggesting that the inhibition was not due to BSA (FIG. 7).

FIG. 7 shows that BSA does not elicit ERK phosphorylation. C6 glial cells were treated with increasing concentrations of BSA alone for 30 min. Resulting lysates (containing 60 μg of total protein) were then subjected to SDS-PAGE and Western blot analysis for the evaluation of ERK phosphorylation. The data reveal that BSA treatment by itself failed to alter ERK phosphorylation levels. The upper blot and lower blot depicts ERK phosphorylation and total ERK protein, respectively, from a single independent experiment. The bar graph (upper panel), however, represents the densitometric analysis from two independent studies, and is presented as signal intensity relative to that seen in the sham (vehicle-treated) control.

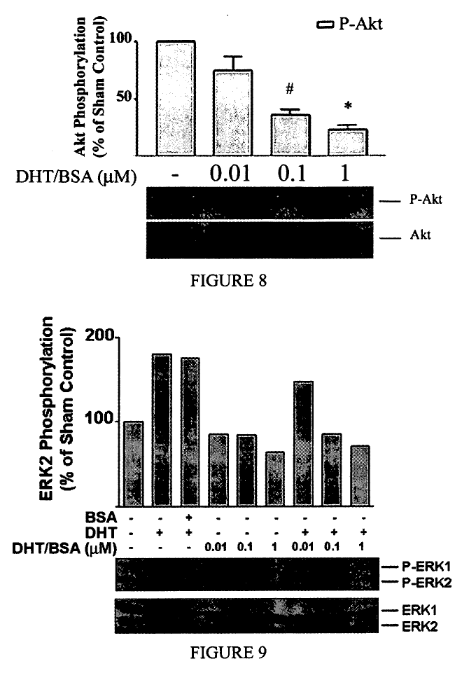

Activation of the membrane androgen receptor results in a dose-dependent decrease in the phosphorylation of Akt. In order to assess if the activation of the putative membrane androgen receptor influenced another growth and/or survival promoting signal transduction pathway in a similar manner to what was observed with the MAPK pathway, the effect of DHT-BSA on the downstream effector of the PI-3 kinase pathway, Akt, was evaluated. DHT-BSA, at concentrations of 0.01, 0.1 and 1 μM resulted in a dose-dependent suppression of Akt phosphorylation (FIG. 8).

FIG. 8 shows that DHT-BSA treatment suppressed phospho-Akt levels in C6 glial cells. C6 glial cells were treated with DHT/BSA (0.01, 0.10, and 1 μM) for 30 min. Resulting lysates (containing 60 μg of total protein) from the various treatment groups were subjected to SDS-PAGE and Western blot analysis for the evaluation of Akt phosphorylation. Treatment of C6 glial cells with DHT-BSA resulted in a dose dependent decrease in phospho-Akt levels. The data demonstrate that DHT-BSA inhibits basal Akt phosphorylation in a dose-dependent manner. The upper blot and lower blot depict Akt phosphorylation and total Akt protein, respectively, from a single independent experiment. The bar graph (upper panel), however, represents the densitometric analysis and statistical evaluation of data from three independent studies, and is presented as signal intensity relative to that seen in the sham (vehicle-treated) control. Statistical significance was determined using one-way ANOVA, followed by Tukey's post-hoc analysis for group differences (*=p<0.001; #=p<0.01).

DHT-BSA blocks DHT-induced ERK phosphorylation in a dose-dependent manner. In view of the inhibitory effects of DHT-BSA on ERK and Akt phosphorylation, and the stimulatory consequence of activating the intracellular androgen receptor (using DHT), the inventors determined if the putative membrane androgen receptor would inhibit the classical AR-mediated induction of ERK phosphorylation. As such, DHT and DHT-BSA were co-applied to the C6 glial cells for 30 min and found that DHT-BSA blocked the effect of DHT (10 nM) on ERK phosphorylation at all concentrations tested (0.01, 0.1, 1 μM), and in a dose dependent fashion (FIG. 9).

FIG. 9 shows that DHT-BSA blocks DHT-induced ERK phosphorylation. To determine if DHT/BSA competitively blocks DHT's effect on the MAPK, C6 glial cells were treated with DHT-BSA at various concentrations (0.01, 0.1, and 1.0 μM) for 30 min. in the presence/absence of DHT (10 nM, a concentration that effectively and reproducibly elicits ERK phosphorylation). As a control, BSA (1 μM) was included. Resulting lysates (containing 60 μg of total protein) were then subjected to SDS-PAGE and Western blot analysis for the evaluation of ERK phosphorylation. The data show that DHT-BSA effectively prevented the effect of DHT on ERK phosphorylation. The upper blot and lower blot depict ERK phosphorylation and total ERK protein, respectively, from a single independent experiment. The bar graph (upper panel), however, represents the densitometric analysis from two independent studies, and is presented as signal intensity relative to that seen in the sham (vehicle-treated) control. These findings support the idea that DHT may modulate the MAPK through at least two, competing pathways.

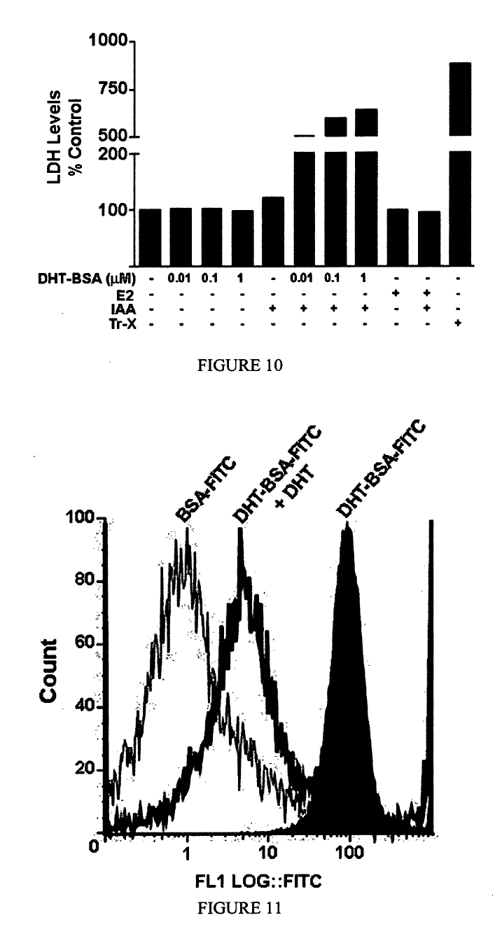

C6 glial cells express binding sites for DHT-BSA on the cell surface. In order to determine if DHT-BSA binds to a specific site on the cell surface (indicative of a membrane-associated androgen receptor), the binding of the fluorescently-labeled DHT-BSA (DHT-BSA-FITC) in C6 glial cells was evaluated. Non-“fixed” and non-permeabilized C6 glial cells were treated with DHT-BSA-FITC (50 μM) in the presence or absence of 20-fold molar excess of DHT (1 mM). Incubation of the cells with BSA-FITC alone for 30 min at 4° C. provided a measure of non-specific binding. The fluorescence-intensity histograms (FIG. 10) obtained through flow cytometric analysis revealed the presence of specific, DHT-displaceable binding sites on the cell surface of the C6 glial cells.

FIG. 10 is a graph that shows that DHT-BSA exacerbates iodoacetic acid (IAA) induced cell death of C6 glial cells. In this assay, increasing concentrations of DHT-BSA alone did not result in cellular damage as assessed by LDH release into the media. However, when superimposed with a low level insult (IAA, 10 μM), DHT-BSA significantly increased the toxicity associated with IAA. Triton-X (Tr-X, 0.9%)-induced lysis of cells served as the positive control for LDH release. Estradiol (E2, 10 μM) treatment of cells served as the positive control for protection against IAA-induced LDH release. Data are representative of two independent studies. The effect shown in FIG. 10, is in sharp contrast to the protective effect of DHT reported previously, and supports further the observation that two, potentially competing, mechanisms regulate the effects of androgens on cell viability. In all studies that used the membrane impermeable androgen, the DHT-BSA preparation was filtered using a 10 kDa nominal cut-off column prior to application of this steroid hormone conjugate, to ensure that the effects of DHT-BSA were not attributed to, or confounded by the presence of free (unconjugated) DHT. Stevis, et al., (Stevis P E, Deecher D C, Suhadolnik L, Mallis L M, Frail D E 1999 Differential effects of estradiol and estradiol-BSA conjugates. Endocrinology 140:5455-8) have described that the commercially available preparation of estradiol-conjugated to BSA (E2-BSA), for example, was contaminated with some free estradiol. These studies shows that any data obtained with this DHT-BSA compound did not suffer from any drawbacks of the design of the study. Further, since the effect of “free” DHT was in the opposite “direction” of that which resulted from application of DHT-BSA, the results demonstrate that the specific effect of DHT-BSA was not due to the contamination of the BSA-conjugated hormone with free hormone.

FIG. 11 shows that DHT-BSA binds to specific sites on the cell surface of C6 glial cells. To determine if DHT-BSA binds to surface (plasma membrane) receptors, C6 glial cells were treated with DHT-BSA-FITC (50 μM) for 30 min. in the presence/absence of a 20-fold molar excess of DHT (1 mM). Samples were washed and analyzed using flow cytometric analysis. The fluorescence histogram, depicting increasing fluorescence intensity on the x-axis, and cell number on the y-axis, describes significant labeling of cells with DHT-BSA-FITC. This labeling appeared to be displaced by DHT. The peak on the extreme left reflects the amount of fluorescence signal obtained when cells were incubated with BSA-FITC alone, representing non-specific binding. The data are representative of three independent studies.

In this study, it was shows that androgens regulate the activity of two signal transduction pathways, the MAPK and the PI-3K/Akt signaling pathways, in C6 glial cells. The existence of immunoreactive androgen receptors (AR-B and AR-A) in the C6 glial cells (FIG. 1), coupled with the fact that DHT-induced ERK phosphorylation was inhibited by the pharmacological antagonist of the classical androgen receptor (FIG. 2) supported the involvement of this “classical” receptor in regulating cell signaling. However, the opposite effect of DHT-BSA on ERK (FIG. 4) and Akt (FIG. 8) phosphorylation suggested that androgens may also regulate cell signaling via a distinct, and potentially, competing receptor mechanism. Supporting this further was the finding that the inhibitory effect of DHT-BSA on ERK phosphorylation was not blocked by the classical androgen receptor antagonist, flutamide (FIG. 5), unlike the effects of DHT (FIG. 2). In addition, analysis of the binding of fluorescently-labeled DHT-BSA to live C6 glial cells revealed specific, DHT-displaceable binding sites on the cell surface (FIG. 10).

In order to exclude the possibility that chemical modification of the parent compound, DHT, was responsible for the observed effects of DHT-BSA, several important controls were performed. First, the inventors made sure that the carboxymethyloxime (CMO) “linker” that enables the attachment of BSA to DHT, did not alter the ability of DHT to regulate cell signaling. FIG. 6 shows that DHT:CMO elicited the same dose-dependent regulation of ERK phosphorylation as did the parent compound (FIG. 2). It is also unlikely that the effect of DHT-BSA was due to the presence of “free” DHT present in the DHT-BSA preparation, since the effect of DHT-BSA was opposite to that observed with DHT. Nevertheless, steps were taken to ensure that no free DHT was present in the DHT-BSA solution. This was achieved by pre-filtering the DHT-BSA using a 30 kDa nominal cut-off column. The retentate was then eluted and used in the studies. All data shown were derived from studies in which the filtered DHT-BSA was used. Further, no differences were observed between the effects of filtered DHT-BSA and unfiltered DHT-BSA (data not shown). Finally, to ensure that BSA by itself was not responsible for the effect of DHT-BSA, it was tested alone. Testing increasing concentrations of BSA, no differences in ERK phosphorylation relative to controls (FIG. 7) was found.

Interestingly, while low concentrations of DHT elicited ERK and Akt phosphorylation, higher concentrations failed to do so (FIGS. 2 and 3). At higher concentrations, DHT may also bind the membrane androgen receptor, resulting in activation of an antagonistic mechanism reducing and/or preventing the induction of ERK phosphorylation via the classical receptor. This result is consistent with the dose response data depicted in FIGS. 2 and 4. That is, the inhibitory effect of DHT-BSA on either ERK or Akt phosphorylation becomes evident at the 0.1 μM concentrations, and only at this same concentration (or higher) does DHT not activate ERK phosphorylation. Further, FIG. 9 also demonstrates that DHT-BSA—induced activation of the putative membrane androgen receptor inhibits the ERK-inducing effects of DHT. Altogether, these data support the existence of two, competing mechanisms through which DHT regulates cell signaling.

Membrane receptors have also been proposed for estrogen and progesterone, but only the membrane progesterone receptor has been successfully cloned. Zhu and colleagues (17) characterized a novel membrane-associated progesterone receptor that appears not to exhibit the stereotypical modular structure seen with other members of the steroid hormone receptor superfamily, but instead, contains a seven transmembrane spanning domain. As such, this membrane progesterone receptor was reported to be coupled to the Gi/o class of G-proteins. Based upon this observation, the inhibitory effects of DHT-BSA on ERK and Akt phosphorylation could also be regulated through Gi/o. To confirm these observations, the inventors evaluated whether pertussis toxin (a Gi/o inhibitor) prevented the effect of DHT-BSA. Pertussis toxin (50 μM) failed to prevent DHT-BSA—mediated suppression of either ERK or Akt phosphorylation (data not shown), as such, the novel membrane androgen receptor was not coupled to the Gi/o class of G-proteins, at least in the C6 glial cell model.

Activation of the ERK/MAPK pathway is associated with various cellular responses, including the induction of cell differentiation, increased cell growth/proliferation, as well as the regulation of cell viability (27, 28). Similarly, the activation of the PI-3K/Akt signaling pathway results in numerous effects on the cell, including the regulation of cell growth, motility, and survival (29). Various studies have shown that, depending on the cell type, androgens can either cause a decrease or an increase in phospho-ERK levels, (5, 22-24, 30, 31), and as a consequence, may result in varied cellular responses. In the brain, this variability in androgen function has also been observed such that depending on the experimental model used or region of the brain evaluated, androgens can exert either protective influences (32, 33), or be damage-promoting (34). For example, in a kainic acid model of hippocampal injury, DHT was found to reduce the amount of hippocampal neuron damage (33), whereas in a middle cerebral artery occlusion (MCAO) model of stroke, elevated androgen levels were associated with greater amounts of cortical cell death (34). This discrepancy may be related to the relative abundance of the classical intracellular/intranuclear androgen receptor and the membrane androgen receptor identified here. Specifically, the protective effects of androgens may be seen only under conditions where the classical androgen receptor predominates, resulting in an increased activation of ERK and/or Akt, which in turn, would favor the promotion of cell survival. In contrast, if the membrane androgen receptor predominates, one might predict that elevated androgens may result in increased vulnerability to insult or injury due to the suppression of neuroprotective signaling pathways.

These data support the existence of a novel membrane-associated AR in glial cells, in addition to the classical intracellular androgen receptor. Further, the data show the existence of two, potentially competing, pathways in a given cell or tissue. Thus, the ratio of one receptor type over another may be used to predict whether androgens are beneficial or detrimental, and as such, could help explain existing discrepancies as to whether androgens are protective or damage-inducing. The present invention may be used to identify, characterize, evaluate and design appropriate therapeutic compounds and regimens that employ androgens and androgen agonists that have a reduced or no entry into the cell for the treatment of various diseases of the brain and other cell types that express the mew membrane AR or both the membrane AR and the “classic” (cytoplasmic or intracellular) AR.

In conclusion, the present invention for the first time supports the observation that a membrane androgen may indeed exist and that it differs structurally and functionally from the classical intracellular androgen receptor. In the system and methods taught herein, the membrane and intracellular androgen receptors demonstrate that two different entities and mechanisms for activation exist in, neuronal cells and the brain.

It was also found that androgens can elicit two different effects depending on which androgen receptor is activated. Furthermore, the present invention may be used to explain why androgens have been reported to be either good or bad and takes this observation further, to provide a rapid, efficient method for detecting and determining the presence and/or absence of the two receptors in real-time. The detection of the two receptors may also be as a ratio, which may be used, e.g., to make a determination of the cell type or status of the cell (healthy, injured, diseased), and the effect of therapy, e.g., the relative proportion of the good receptor and the bad receptor may change.

Thus, depending on the relative ratio of the membrane androgen receptor to the classical intracellular androgen receptor, the outcome consequent to androgen therapy may be different. Androgen receptor status may be used to predict the therapeutic efficacy of androgens in a particular patient and/or the effect of on-going therapy on the prognosis for disease and its effect.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Mooradian A D, Morley J E, Korenman S G 1987 Biological actions of androgens. Endocr Rev 8:1-28
2. Mangelsdorf D J, Thummel C, Beato M, et al. 1995 The nuclear receptor superfamily: the second decade. Cell 83:835-9
3. Baron S, Manin M, Beaudoin C, et al. 2004 Androgen receptor mediates non-genomic activation of phosphatidylinositol 3-OH kinase in androgen-sensitive epithelial cells. J Biol Chem 279:14579-86
4. Sun M, Yang L, Feldman R I, et al. 2003 Activation of phosphatidylinositol 3-kinase/Akt pathway by androgen through interaction of p85alpha, androgen receptor, and Src. J Biol Chem 278:42992-3000
5. Peterziel H. Mink S, Schonert A, Becker M, Klocker H, Cato A C 1999 Rapid signalling by androgen receptor in prostate cancer cells. Oncogene 18:6322-9
6. Wilson C M, McPhaul M J 1994 A and B forms of the androgen receptor are present in human genital skin fibroblasts. Proc Natl Acad Sci USA 91:1234-8
7. Wilson C M, McPhaul M J 1996 A and B forms of the androgen receptor are expressed in a variety of human tissues. Mol Cell Endocrinol 120:51-7
8. Gao T, McPhaul M J 1998 Functional activities of the A and B forms of the human androgen receptor in response to androgen receptor agonists and antagonists. Mol Endocrinol 12:654-63
9. Liegibel U M, Sommer U, Boercsoek I, et al. 2003 Androgen receptor isoforms AR-A and AR-B display functional differences in cultured human bone cells and genital skin fibroblasts. Steroids 68:1179-87
10. Papakonstanti E A, Kampa M, Castanas E, Stournaras C 2003 A rapid, nongenomic, signaling pathway regulates the actin reorganization induced by activation of membrane testosterone receptors. Mol Endocrinol 17:870-81
11. Shah C, Modi D, Gadkar S, Sachdeva G, Puri C 2003 Progesterone receptors on human spermatozoa. Indian J Exp Biol 41:773-80
12. Toran-Allerand C D, Guan X, MacLusky N J, et al. 2002 ER-X: a novel, plasma membrane-associated, putative estrogen receptor that is regulated during development and after ischemic brain injury. J Neurosci 22:8391-401
13. Sakamoto H, Ukena K, Takemori H, Okamoto M, Kawata M, Tsutsui K 2004 Expression and localization of 25-Dx, a membrane-associated putative progesterone-binding protein, in the developing Purkinje cell. Neuroscience 126:325-34
14. Pappas T C, Gametchu B, Watson C S 1995 Membrane estrogen receptors identified by multiple antibody labeling and impeded-ligand binding. Faseb J 9:404-10
15. Marquez D C, Pietras R J 2001 Membrane-associated binding sites for estrogen contribute to growth regulation of human breast cancer cells. Oncogene 20:5420-30
16. Rambo C O, Szego C M 1983 Estrogen action at endometrial membranes: alterations in luminal surface detectable within seconds. J Cell Biol 97:679-85
17. Zhu Y, Rice C D, Pang Y, Pace M, Thomas P 2003 Cloning, expression, and characterization of a membrane progestin receptor and evidence it is an intermediary in meiotic maturation of fish oocytes. Proc Natl Acad Sci USA 100:2231-6
18. Heinlein C A, Chang C 2002 The roles of androgen receptors and androgen-binding proteins in nongenomic androgen actions. Mol Endocrinol 16:2181-7
19. Benten W P, Lieberherr M, Stamm O, Wrehlke C, Guo Z, Wunderlich F 1999 Testosterone signaling through internalizable surface receptors in androgen receptor-free macrophages. Mol Biol Cell 10:3113-23
20. Benten W P, Becker A, Schmitt-Wrede H P, Wunderlich F 2002 Developmental regulation of intracellular and surface androgen receptors in T cells. Steroids 67:925-31
21. Braun A M, Thomas P 2004 Biochemical characterization of a membrane androgen receptor in the ovary of the atlantic croaker (Micropogonias undulatus). Biol Reprod 71:146-55
22. Somjen D, Kohen F, Gayer B, Kulik T, Knoll E, Stern N 2004 Role of putative membrane receptors in the effect of androgens on human vascular cell growth. J Endocrinol 180:97-106
23. Benten W P, Guo Z, Krucken J, Wunderlich F 2004 Rapid effects of androgens in macrophages. Steroids 69:585-90
24. Estrada M, Espinosa A, Muller M, Jaimovich E 2003 Testosterone stimulates intracellular calcium release and mitogen-activated protein kinases via a G protein-coupled receptor in skeletal muscle cells. Endocrinology 144:3586-97
25. Singh M, Setalo G, Jr., Guan X, Warren M, Toran-Allerand C D 1999 Estrogen-induced activation of mitogen-activated protein kinase in cerebral cortical explants: convergence of estrogen and neurotrophin signaling pathways. J Neurosci 19:1179-88
26. Lowry O H, Rosebrough N J, Farr A L, Randall R J 1951 Protein measurement with the Folin phenol reagent. J Biol Chem 193:265-75
27. Marshall C J 1995 Specificity of receptor tyrosine kinase signaling: transient versus sustained extracellular signal-regulated kinase activation. Cell 80:179-85
28. Stanciu M, DeFranco D B 2002 Prolonged nuclear retention of activated extracellular signal-regulated protein kinase promotes cell death generated by oxidative toxicity or proteasome inhibition in a neuronal cell line. J Biol Chem 277:4010-7
29. Cantley L C 2002 The phosphoinositide 3-kinase pathway. Science 296:1655-7
30. Kayampilly P P, Menon K M 2004 Inhibition of extracellular signal-regulated protein kinase-2 phosphorylation by dihydrotestosterone reduces follicle-stimulating hormone-mediated cyclin D2 messenger ribonucleic acid expression in rat granulosa cells. Endocrinology 145:1786-93
31. Bell W C, Myers R B, Hosein T O, Oelschlager D K, Grizzle W E 2003 The response of extracellular signal-regulated kinase (ERK) to androgen-induced proliferation in the androgen-sensitive prostate cancer cell line, LNCaP. Biotech Histochem 78:11-6
32. Ramsden M, Nyborg A C, Murphy M P, et al. 2003 Androgens modulate beta-amyloid levels in male rat brain. J Neurochem 87:1052-5
33. Ramsden M, Shin T M, Pike C J 2003 Androgens modulate neuronal vulnerability to kainate lesion. Neuroscience 122:573-8
34. Yang S H, Liu R, Wen Y, et al. 2005 Neuroendocrine mechanism for tolerance to cerebral ischemia-reperfusion injury in male rats. J Neurobiol 62:341-51
35. Finley S K, Kritzer M F 1999 Immunoreactivity for intracellular androgen receptors in identified subpopulations of neurons, astrocytes and oligodendrocytes in primate prefrontal cortex. J Neurobiol 40:446-57
36. Garcia-Ovejero D, Veiga S, Garcia-Segura L M, Doncarlos L L 2002 Glial expression of estrogen and androgen receptors after rat brain injury. J Comp Neurol 450:256-71

What is claimed is:

1. A method for selecting a drug candidate, comprising the steps of:
comparing the binding specificity of one or more candidate drugs to a membrane androgen receptor and an intracellular androgen receptor, wherein a difference in drug candidate binding is indicative of differential receptor binding.

2. The method of claim 1, wherein the binding specificity is measured by flow cytometry, protein kinase activation, protein phosphorylation, gene expression, mRNA stability, protein expression, antibody binding, scintillation counting, cell count, cell division, cell viability or cell apoptosis.

3. The method of claim 1, wherein the membrane androgen receptor is measured using a non-internalized androgen bound to a non-internalizable moiety.

4. The method of claim 1, wherein the membrane androgen receptor is measured using a DHT-BSA, a DHT-bead, DHT-conjugated to a membrane-impermeable moiety, or a DHT-substrate.

5. The method of claim 1, wherein the intracellular androgen binding agent comprises one or more androgen binding compound selected from testosterone, dihydrotestosterone, active metabolites of testosterone, and synthetic derivatives of testosterone such as testosterone propionate, testosterone cypionate, fluoxymesterone, flutamide and combinations thereof.

6. The method of claim 1, wherein activation of the membrane androgen receptor modulates cell survival and/or cell death.

7. The method of claim 1, wherein activation of the membrane androgen receptor occurs independent of the intracellular androgen receptor.

8. The method of claim 1, wherein activation of the membrane androgen receptor promotes the viability of brain cells.

9. The method of claim 1, further comprising the step of determining a relative ratio of the membrane androgen receptor to the classical intracellular androgen receptor, wherein the ratio of membrane to intracellular androgen receptor is used to predict the outcome of androgen therapy.

10. The method of claim 1, further comprising the step of screening for the relative abundance of the membrane androgen receptor to identify patients that can receive androgen therapy safely versus those for which androgen therapy is contraindicated.

11. A kit comprising in one or more vials, a non-internalizable androgen receptor binding agent, a labeled displaceable androgen and an intracellular androgen receptor binding agent, wherein the kit is used to measure the difference in the levels of a membrane androgen receptor and an intracellular binding receptor.

12. The kit of claim 11, wherein the non-internalizable androgen receptor binding agent comprises a C-19 steroid bound to a bead, an albumin-coated bead, an albumin protein, a charged chemical moiety, or a glass bead.

13. The kit of claim 11, further comprising a non-internalizable, labeled-displaceable androgen.

14. The kit of claim 11, further comprising a displaceable androgen labeled a detectable marker selected from a radio-opaque material, a radioactive label, an enzymatic label, an epitope tag, a poly-His tag, a bead, a magnetic bead, a metal, or a fluoresent label such as 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, a Lanthanide Chelate, a Lanthanide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

15. A method for diagnosing the relative health of a cell comprising comparing the ratio of membrane to intracellular androgen receptors, wherein a change in the ratio indicates a change in cell viability.

16. A method for selecting patient for a clinical trial comprising the steps of:
determining a ratio of a membrane androgen receptor to an intracellular androgen receptor from a candidate patient sample; and
optionally including or excluding the candidate patient from the trial if the ratio indicates a sensitivity to androgen therapy.

17. The method of claim 16, wherein the sensitivity is to intracellular androgen receptor activation, intracellular androgen receptor inactivation, membrane androgen receptor activation, membrane androgen receptor inactivation and combinations thereof.

18. The method of claim 16, wherein the patient is suspected of having a disease associated with or related pathogenetically to androgen receptor activation and wherein the patient is tested for the ratio of preferential binding of a candidate agent to a membrane androgen receptor or an intracellular androgen receptor.

19. A method for identifying one or more candidate agents from a pool of agents comprising the steps of:
detecting the effect of one or more agents from a pool of agents on membrane androgen receptor binding, wherein a change in the level of binding is indicative of competition of the membrane androgen receptor and is used to further identify the candidate agents.

20. The method of claim 19, further comprising the step of detecting the binding of the one or more candidate agents to the intracellular androgen receptor binding.

21. The method of claim 19, further comprising the step of detecting the binding of the one or more candidate agents to the intracellular androgen receptor binding; and
selecting further those candidate agents that have higher bind to the membrane androgen receptor than to the intracellular androgen receptor.

22. A method treating a disease associated with or related pathogenetically to androgen receptor activation comprising preferentially binding a membrane androgen receptor or an intracellular androgen receptor.

23. The method of claim 22, wherein the membrane androgen receptor is preferentially activated over the intracellular androgen receptor.

24. The method of claim 22, wherein the intracellular androgen receptor is preferentially activated over the membrane androgen receptor.

25. The method of claim 22, wherein the disease is an age-related disease comprises brain cell apoptosis.

26. A composition for treating a disease associated with or related pathogenetically to androgen receptor activation comprising a composition that binds preferentially to a membrane androgen receptor or an intracellular androgen receptor.

27. The composition of claim 26, wherein the composition that binds preferentially to a membrane androgen receptor is selected from a protein, nucleic acid, carbohydrate, lipid, fatty acid, bead, a membrane-impermeable moiety, a substrate and combinations thereof that are conjugated to a testosterone, dihydrotestosterone, active metabolites of testosterone, and synthetic derivatives of testosterone such as testosterone propionate, testosterone cypionate, fluoxymesterone, flutamide and combinations thereof.

28. The composition of claim 26, wherein the composition comprises one or more compositions selected from a membrane androgen receptor is selected from a DHT-BSA, a DHT-bead, DHT-conjugated to a membrane-impermeable moiety, or a DHT-substrate; and
one or more intracellular androgen receptor binding compounds selected from testosterone, dihydrotestosterone, active metabolites of testosterone, and synthetic derivatives of testosterone such as testosterone propionate, testosterone cypionate, and fluoxymesterone, wherein the ratio of the membrane versus intracellular androgen receptor binding agent is modulated to treat the disease.

29. A method for treating brain cells associated with or related pathogenetically to androgen receptor activation comprising preferentially binding an active agent to a membrane androgen receptor.

30. The method of claim 29, wherein the active agent comprises a protein, nucleic acid, carbohydrate, lipid, fatty acid, bead, a membrane-impermeable moiety, a substrate and combinations thereof conjugated to a testosterone, dihydrotestosterone, active metabolites of testosterone, and synthetic derivatives of testosterone such as testosterone propionate, testosterone cypionate, fluoxymesterone, flutamide and combinations thereof.

31. The method of claim 29, wherein the active agent inactivates the membrane androgen receptor.

32. The method of claim 29, wherein the active agent binds specifically to a membrane androgen receptor.

33. The method of claim 29, wherein the active agent comprises both an intracellular androgen receptor binding agent selected from testosterone, dihydrotestosterone, active metabolites of testosterone, and synthetic derivatives of testosterone such as testosterone propionate, testosterone cypionate, fluoxymesterone, flutamide and combinations thereof; and
a protein, nucleic acid, carbohydrate, lipid, fatty acid, bead, a membrane-impermeable moiety, a substrate and combinations thereof conjugated to a testosterone, dihydrotestosterone, active metabolites of testosterone, and synthetic derivatives of testosterone such as testosterone propionate, testosterone cypionate, fluoxymesterone, flutamide and combinations thereof,
wherein the ratio of the membrane versus intracellular androgen receptor binding agent is modulated to treat the disease.

34. The method of claim 33, wherein the ratio of intracellular to membrane androgen receptor binding agent is from between about 1:100 to 100:1.

35. A composition for treating a disease associated with or related pathogenetically to a membrane-associated androgen receptor comprising a pharmaceutically effective amount of an androgen receptor binding agent conjugated to a moiety that prevents or reduces the entry of the composition into a cell.

36. The composition of claim 35, wherein the moiety that prevents or reduces the entry of the composition into the cell is selected from a protein, lipid, fatty acid, carbohydrate, a nucleic acid and combinations thereof.
